(19) United States
(12) Patent Application Publication
(10) Pub. No.: US 2026/0287474 A1
(43) Pub. Date: Sep. 24, 2026

(54) A SAMPLING DEVICE AND A SAMPLING SYSTEM FOR SAMPLING OF A LIQUID

(71) Applicant: Ascilion AB, Kista (SE)

(72) Inventor: Mikael Hillmering, Sollentuna (SE)

(73) Assignee: Ascilion AB, Kista (SE)

(21) Appl. No.: 19/168,223

(22) PCT Filed: Mar. 27, 2024
(86) PCT No.: PCT/SE2024/050280

§ 371 Date: Sep. 23, 2025

(30) Foreign Application Priority Data
Mar. 27, 2023 (SE) 2350344-4

(51) Int. Cl. G01N 1/10

(52) U.S. Cl. CPC G01N 1/10

(57) ABSTRACT

A sampling device comprising a microneedle array assembly comprising at least one microneedle, a body that together with the microneedle array assembly defines a compartment in fluid connection with the microneedles of the microneedle array assembly, a first fluid port mouthing into the compartment and having a first diameter d1, and a second fluid port in fluid connection with a suction tube having an inner diameter d2 and extending into the compartment, such that an open end of the suction tube is at a distance s from the microneedle array assembly.

### TECHNICAL FIELD

The present invention relates to sampling device and more specifically to a sampling device with a microneedle array for sampling of a liquid.

### BACKGROUND

In recent years there has been an increased interest related to sampling of liquids and bodily fluids with a microneedle array (MNA). However, this type of sampling is often associated with a number of problems. In order to promote flow of the liquid through the microneedle array (MNA) it is often suggested to subject the MNA to a sub pressure such that the liquid is drawn through the microneedles to a sample area of the MNA. However, when the surrounding air pressure enters the sample area when the sampling device is removed from the test specimen it is quite common that the sampled liquid transforms into a foam due to the air that flows into the MNA from the outside due to the sub pressure. It is therefore of great interest to provide a solution that minimizes the formation of foam and provides an efficient transfer of the sampled liquid to a sample container for further analysis.

It is therefore an object of the present invention to suggest an improved sampling device, and sampling system, which mitigates, or eliminates, the above problems.

### SUMMARY

The objective of the invention is achieved by means of a sampling device comprising a microneedle array assembly comprising at least one microneedle, a body that together with the microneedle array assembly defines a compartment in fluid connection with the microneedles of the microneedle array assembly, a first fluid port mouthing into the compartment and having a first diameter d1, and a second fluid port in fluid connection with a suction tube having an inner diameter d2 and extending into the compartment, such that an open end of the suction tube is at a distance s from the microneedle array assembly.

According to one embodiment, the suction tube is a flexible tube. This allows a precise arrangement of the suction tube such that the same abuts the sample area of a MNA.

According to one embodiment, wherein d1>d2. This allows for example the use of a different sub pressure to promote transfer of the sampled liquid through the suction tube.

According to one embodiment, the d1>5*d2.

According to one embodiment, the distance s is smaller than 4*d2. This allows an efficient suction of the sampled liquid through the open end of the suction tube.

According to one embodiment, the suction tube is arranged within the first fluid port. This allows a very compact solution that requires only one opening to the compartment from the outside.

According to one embodiment, the sampling device further comprises fastening means for sealed removable connection of the body to the microneedle array assembly. This allows the body to be used as a suction device without the MNA mounted on the body.

The objective of the invention is also achieved with a sampling system for sampling a fluid having a sampling device according to embodiments as disclosed herein, and a sealed sample container in fluid connection with the suction tube via the second fluid port for receiving the sampled fluid, wherein the sample container comprises a fluid port. This allows for efficient transfer of the sampled liquid to the sample container.

According to one embodiment, the fluid port of the sample container is in fluid connection to a vacuum pump for applying a pressure P2 the sample container, and wherein the first fluid port of the sampling device is in fluid connection with a further vacuum pump for applying a pressure P1 to said first fluid port. This allows using separate pressures in order to promote liquid flow from the sample area to the sample container in an efficient way.

According to one embodiment, the P2≤P1<P0 and P0 is the atmospheric pressure outside the sampling device. This allows a very efficient transport of liquid from the MNA to the sample container.

According to one embodiment, the fluid port of the sample container is in fluid connection with a first port of a three-port valve, wherein the first fluid port of the sampling device is in fluid connection with a second port of the three-port valve, and a vacuum pump is in fluid connection with a third port of the three-port valve for applying a pressure P1 to one of the first or the second ports of the three-port valve. This allows a minimum number of valves to be used, while still obtaining the advantages of the solution.

According to one embodiment, P1<P0 where P0 is the atmospheric pressure outside the sampling device.

Further objects and advantages may be found in the detailed description.

### BRIEF DESCRIPTION OF THE DRAWINGS

FIG. 1 is a schematic cross section of a sampling device according to the present invention,

FIG. 2 is a schematic cross section of a sampling device according to the present invention,

FIG. 3 is a schematic cross section of a sampling system according to the present invention,

FIG. 4 is a schematic block diagram of a sampling system according to the present invention,

FIG. 5 is a schematic perspective view of a sampling system according to the present invention,

FIG. 6 is a schematic perspective view from above of the sampling system disclosed in FIG. 5, and

FIG. 7 is a cross sectional view of the sampling system disclosed in FIG. 6 along line A-A.

### DETAILED DESCRIPTION

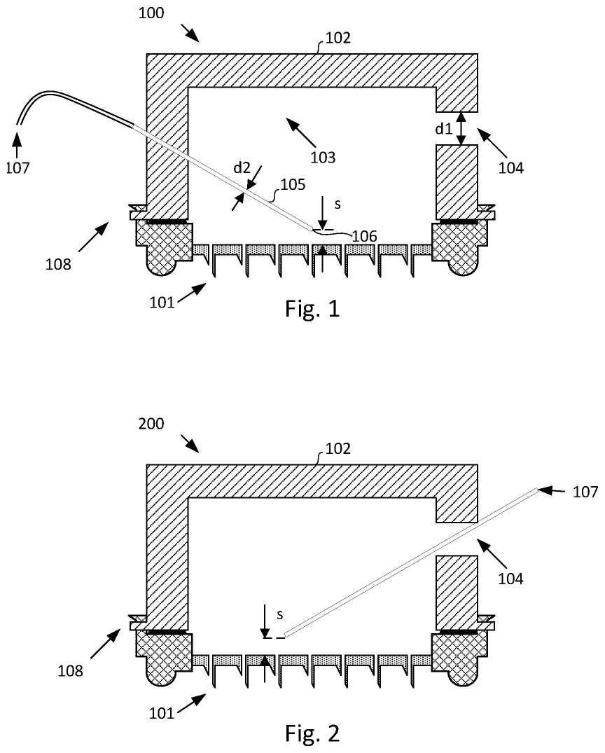

FIG. 1 shows cross sectional view of a sampling device according to the present invention. The sampling device, generally designated 100, comprises a microneedle array assembly 101 comprising at least one microneedle, and a body 102 that together with the microneedle array assembly 101 defines a compartment 103 in fluid connection with the microneedles of the microneedle array assembly 101. The sampling device comprises a first fluid port 104 mouthing into the compartment 103 and having a first diameter d1, and a second fluid port 107 in fluid connection with a suction tube 105 having an inner diameter d2 and extending into the compartment 103. Such that an open end 106 of the suction tube 105 is at a distance s from the microneedle array assembly 101.

The suction tube 105 is a flexible tube, which facilitates that it abuts the sampling area of the MNA. Preferably, d1>d2, and most preferably d1>5*d2. This provides an efficient suction action by the open end of the suction tube.

In order to provide an efficient suction action, the distance s should be smaller than 4*d2. Otherwise, the open end 106 may be too far from the sampled liquid surface in the sampling area. If the distance s is smaller than 4*d2 liquid is transferred to the open end 106 efficiently. The suction action may be further promoted by a slightly flattened open end of the suction tube. Thereby, forming a nozzle in the open end of the suction tube.

Now with reference made to FIG. 2. In which one embodiment of the sampling device, generally designated 200, is illustrated in a cross sectional view. This embodiment differs from the embodiment disclosed in FIG. 1 in that the suction tube 105 is arranged within the first fluid port 104. This allows a compact arrangement that will be further discussed below.

According to embodiments of the present invention, the sample device further comprises fastening means 108 for sealed removable connection of the body 102 to the microneedle array assembly 101. This allows a procedure in which the MNA first penetrates the skin of a test specimen assisted by sub pressure in the sampling device. After the penetration the MNA is removed from the body and the body is placed on top of the penetrated skin area, a subsequent step of providing a sub pressure to the first port causes the sample fluid to form a droplet on the skin of the test specimen. This droplet may then be collected with the suction tube by applying a sub pressure to the second fluid port.

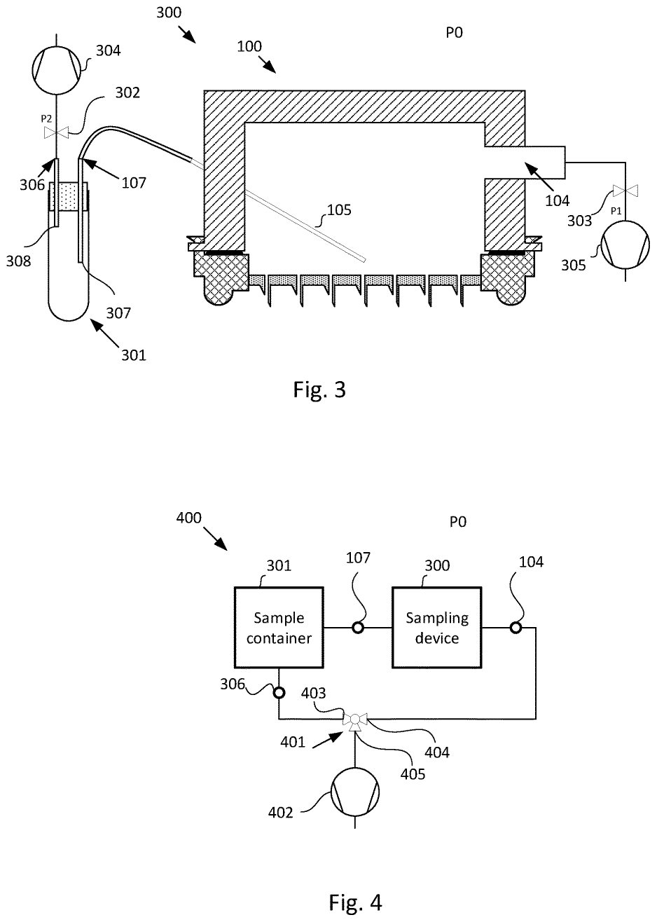

Now with reference made to FIG. 3 in which a sampling system, generally designated 300, according to one embodiment of the present invention is disclosed. The sampling system 300 comprises a sampling device 100 according to embodiments of the present invention as disclosed herein. The sampling system further comprises a sealed sample container 301 in fluid connection with the suction tube 105 via the second fluid port 107 for receiving the sampled fluid, wherein the sample container comprises a fluid port 306.

The fluid port 306 of the sample container is in fluid connection to a vacuum pump 304 for applying a pressure P2 the sample container 301. The first fluid port 104 of the sampling device 100 is in fluid connection with a further vacuum pump 305 for applying a pressure P1 to said first fluid port 104. The vacuum pump 304 is connected to a valve 302 for control of the fluid flow from the suction tube. The further vacuum pump 305 is also connected to the first fluid port 104 via a valve 303. Preferably, the pressures relates to each other as P2<P1<P0 and P0 is the atmospheric pressure outside the sampling device. The fluid port 306 is in fluid contact with a first end 308 of a first tube, which extends a first distance into the sample container. The second fluid port 107 is in fluid contact with a first end 307 of a second tube that extends a second distance into the sample container, wherein the first distance is shorter than the second distance.

Now with reference made to FIG. 4 in which an embodiment of a sampling system 400 is disclosed as a block diagram. In this embodiment, the fluid port 306 of the sample container is in fluid connection with a first port 403 of a three-port valve 401. The first fluid port 104 of the sampling device 100 is in fluid connection with a second port 404 of the three-port valve 401. A vacuum pump 402 is in fluid connection with a third port 405 of the three-port valve 401 for applying a pressure P1 to one of the first or the second ports of the three-port valve 401. Preferably, P1<P0 where P0 is the atmospheric pressure outside the sampling device.

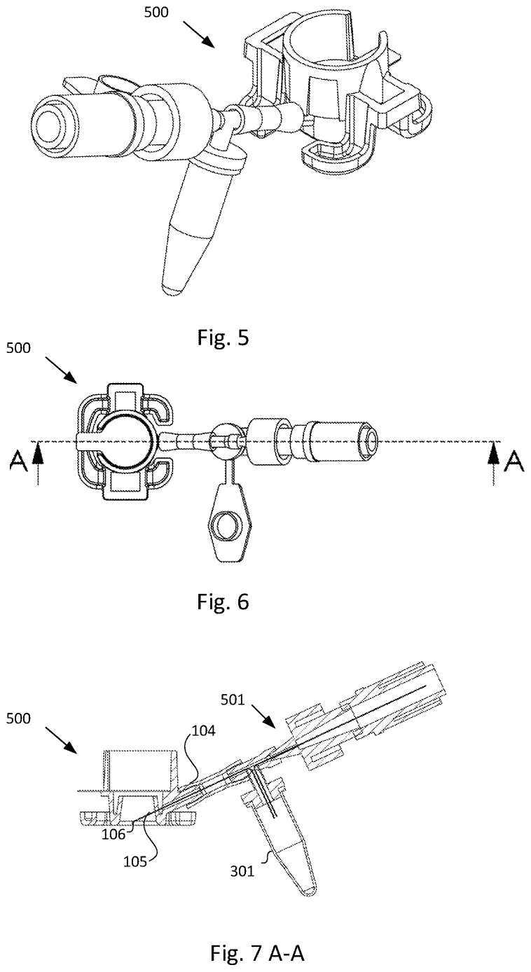

Reference is now made to FIG. 5 to 7, which discloses a body 500 according to one embodiment of the present invention. In FIG. 7, a cross-sectional view along line A-A in FIG. 6 is disclosed. In this embodiment, the suction tube 105 passes through the first fluid port 104 as discussed above with reference made to FIG. 2. The flow through the first fluid port and the second fluid port is adjusted by means of the valve 501. Such that when the valve 501 is fully open, there is a fluid flow through the first fluid port and the second fluid port. Upon closing the valve 501, first, the fluid flow through the first fluid port is restricted and flow through the suction tube 105 is promoted, and upon further closing of the valve 501, the flow through the second fluid port is restricted. This arrangement provides an efficient control of the liquid flow to the sample container 301. Furthermore, it should be noted that the body and the components of the sampling system could be disposables.

1. A sampling device for sampling a liquid, comprising:
a microneedle array assembly comprising at least one microneedle;
a body that together with the microneedle array assembly defines a compartment in fluid connection with the microneedles of the microneedle array assembly;
a first fluid port mouthing into the compartment and having a first diameter d1; and
a second fluid port in fluid connection with a suction tube having an inner diameter d2 and extending into the compartment such that an open end of the suction tube is at a distance s from the microneedle array assembly.

2. The sampling device according to claim 1, wherein the suction tube is a flexible tube.

3. The sampling device according to claim 1, wherein d1>d2.

4. The sampling device according to claim 3, wherein d1>5*d2.

5. The sampling system according to claim 1. wherein the distance s is smaller than 4*d2.

6. The sampling device according to claim 1. wherein the suction tube is arranged within the first fluid port

7. The sampling device according to claim 1, further comprising fastening means for sealed removable connection of the body to the microneedle array assembly.

8. A sampling system for sampling of a fluid, comprising:
a sampling device according to claim 1, a sealed sample container in fluid connection with the suction tube via the second fluid port for receiving the sampled fluid, wherein the sample container comprises a fluid port.

9. The sampling system according to claim 8, wherein the fluid port of the sample container is in fluid connection to a vacuum pump for applying a pressure P2 to the sample container, and wherein the first fluid port of the sampling device is in fluid connection with a further vacuum pump for applying a pressure P1 to said first fluid port.

10. The sampling system according to claim 9, wherein P2≤P1<P0 and P0 is the atmospheric pressure outside the sampling device.

11. The sampling system according to claim 8, wherein the fluid port of the sample container is in fluid connection with a first port of a three-port valve, wherein the first fluid port of the sampling device is in fluid connection with a second port of the three-port valve and a vacuum pump is in fluid connection with a third port of the three-port valve for applying a pressure P1 to one of the first or the second ports of the three-port valve.

12. The sampling system according to claim 11, wherein P1<P0 where P0 is the atmospheric pressure outside the sampling device.
